(19) United States
(12) Patent Application Publication
(10) Pub. No.: US 2004/0161391 A1
(43) Pub. Date: Aug. 19, 2004

(54) Ascorbic acid compounds as bleaching agents

(75) Inventors: Maria Dalko, Gif S/Yvette (FR); Alexandre Cavezza, Tremblay-En-France (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/736,570

(22) Filed: Dec. 17, 2003

Related U.S. Application Data
(60) Provisional application No. 60/437,019, filed on Dec. 31, 2002.

(30) Foreign Application Priority Data
Dec. 18, 2002 (FR) 0216109

(51) Int. Cl. A61K 7/135

(52) U.S. Cl. 424/62

(57) ABSTRACT

The present invention relates to the use of ascorbic acid compounds, especially esters, alone or in a composition, preferably a cosmetic composition, to, e.g., bleach human skin, head hair or other hairs, protect against tanning, and/or reverse the effects of tanning.

### REFERENCE TO PRIOR APPLICATIONS

This application claims priority to U.S. provisional application 60/437,019 filed Dec. 31, 2002, and to French patent application 0216109 filed Dec. 18, 2002, both incorporated herein by reference.

### FIELD OF THE INVENTION

The present invention relates to the use of ascorbic acid compounds, especially esters, alone or in a composition, preferably a cosmetic composition, to, e.g., bleach human skin, head hair or other hairs, protect against tanning, and/or reverse the effects of tanning.

The invention also relates to the use of ascorbic acid compounds, especially esters, for the preparation of a composition, especially a dermatological composition, for depigmenting human skin, head hair or other hairs.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The color of human skin depends on different factors and, in particular, the seasons of the year, race and sex, and is mainly determined by the nature and concentration of melanin produced by the melanocytes. Melanocytes are specialized cells which synthesize melanin by means of specific organelles, the melanosomes. In addition, at different periods in their life, certain individuals develop darker and/or more colored blemishes on the skin and more especially on the hands, making the skin non-uniform. These blemishes are also due to a large concentration of melanin in the keratinocytes at the skin surface.

In the same way, the color of head hair and other hairs is due to melanin. When head hair or other hairs are dark, certain people wish to have them lighter. This is particularly advantageous for hairs which are less visible when they are light than when they are dark.

It is most particularly sought to use harmless topical depigmenting substances which are of good efficacy, in order to treat regional hyper-pigmentations caused by melanocyte hyperactivity, such as idiopathic melasmas, occurring during pregnancy (“pregnancy mask” or chloasma) or during oestro-progestative contraception, localized hyper-pigmentations caused by hyperactivity and proliferation of benign melanocytes, such as senile pigmentation marks known as actinic lentigo, accidental hyper-pigmentations, possibly due to photosensitization or to post-lesional cicatrization, as well as certain leukodermias, such as vitiligo. For the latter, (in which the cicatrizations can result in a scar which gives the skin a whiter appearance), failing being able to repigment the damaged skin, the regions of residual normal skin are depigmented in order to give the skin as a whole a uniform white complexion.

The mechanism for the formation of skin pigmentation, that is to say the formation of melanin, is particularly complex and schematically involves the following main steps:

Tyrosine→Dopa→Dopaquinone→Dopachrome→Melanin

Tyrosinase (monophenol dihydroxyl phenylalanine: oxygen oxidoreductase EC 1.14.18.1) is the essential enzyme involved in this reaction sequence. It especially catalyzes the reaction for the conversion of tyrosine into dopa (dihydroxyphenyl-alanine) by virtue of its hydroxylase activity and the reaction for the conversion of dopa into dopaquinone by virtue of its oxidase activity. This tyrosinase acts only when it is in the mature state, under the action of certain biological factors.

In the epidermis, melanocytes are involved in the epidermal melanin unit that comprises a melanocyte surrounded by about 36 neighbouring keratinocytes. All individuals, without distinction as to phototype, have approximately the same number of melanocytes for a given region of skin. Ethnic differences, in terms of pigmentation, are not due to the number of melanocytes, but to the properties of their melanosomes. The melanosomes are aggregated into complexes and are of small size. They are highly specialized organelles whose sole function is to produce melanin. They arise from the endoplasmic reticulum in the form of spherical vacuoles known as premelanosomes. The premelanosomes contain an amorphous protein substrate, but no melanin-generating enzymes. During maturation of the premelanosome, the amorphous substrate becomes organized into a fibrillar structure oriented in the longitudinal axis of the melanosome. Four stages in the development of melanosomes are distinguished, corresponding to the intensity of melanization. Melanin is deposited uniformly over the inner fibrillar network of the melanosome and the opacity of the organelle increases to the point of saturation. Gradually as melanin is synthesized in the melanosomes, these melanosomes move from the perinuclear region towards the extremity of the dendrites of the melanocytes. Via phagocytosis, the extremity of the dendrites is captured by the keratinocytes, the membranes are degraded and the melanosomes are redistributed in the keratinocytes. The dendritic extensions of the melanocytes thus play an essential role in transferring melanin.

Although the level of melanin varies from one population to another, the amount of tyrosinase does not vary significantly and the level of tyrosinase messenger RNA is identical in black or white skin. Variations in melanogenesis are thus caused by variations either in the activity of tyrosinase or in the capacity of the keratinocytes to phagocytose the melanosomes, or alternatively in the number of dendritic extensions.

A substance is recognized as being depigmenting if it acts directly on the vitality of the epidermal melanocytes in which melanogenesis takes place, for example by modifying the morphology of the dendritic extensions, and/or if it interferes with one of the steps in the biosynthesis of melanin either by inhibiting one of the enzymes involved in melanogenesis or by becoming intercalated as a structural analogue of one of the chemical compounds in the melanin synthesis chain, whereby this chain may then be blocked and thus ensure the depigmentation.

The substances most commonly used as depigmenting agents are, more particularly, hydroquinone and its compounds, in particular its ethers such as hydroquinone monomethyl ether and monoethyl ether. Although they have a certain level of efficacy, these compounds are unfortunately not free of side effects on account of their toxicity, which can make them difficult or even hazardous to use. This toxicity arises from the fact that they interfere with fundamental mechanisms of melanogenesis, by killing cells which then risk disrupting their biological environment and which consequently force the skin to eliminate them by producing toxins.

Thus, hydroquinone is a compound which is particularly irritant and cytotoxic to melanocytes, and whose total or partial replacement has been envisaged by many authors.

Substances have thus been sought which are not involved in the mechanism of melanogenesis, but which act upstream on tyrosinase by preventing its activation, and are consequently much less toxic. Kojic acid is commonly used as tyrosinase-activation inhibitor, this acid complexing the copper present in the active site of this enzyme. Unfortunately, this compound is unstable in solution, which somewhat complicates the manufacture of the composition.

The activity of ascorbic acid or of certain compounds thereof on pigmentation has been known for many years. With the aim of explaining their action on melanogenesis, an inhibitory effect on tyrosinase has been demonstrated for ascorbic acid (J. Soc. Cosmet. Chem., 42, 1991, pp. 361-368). Recently, a study has also shown that magnesium ascorbyl phosphate can have an effect on melanin transfer and can reduce the dendricity of the melanocytes (Pigment. Cell. Res., 13, 2000, pp. 89-98 and pp. 190-192), thus inducing a depigmenting effect. This effect is comparable to that which may be obtained with ferulic acid.

There is still a need for a novel agent for bleaching human skin, head hair and/or other hairs, which is just as effective as the known agents, but which does not have their drawbacks, i.e. which is non-irritant, non-toxic and/or non-allergizing for the skin, while at the same time being stable in a composition, or alternatively which has reinforced action so as to be able to be used in a smaller amount, thus considerably reducing the observed side effects.

### DETAILED DESCRIPTION OF THE PREFERRED EMBODIMENTS

In this regard, the inventors have discovered, surprisingly and unexpectedly, that certain ascorbic acid compounds have good depigmenting activity, even at low concentration, without showing any cytotoxicity. These compounds furthermore have the advantage of acting simultaneously on several components of the pigmentation mechanism. While not bound by theory, they are believed to act by inhibiting the biosynthesis of melanin and by inhibiting the dendricity of the melanocytes, thus reducing the amount of melanin transferred to the neighbouring keratinocytes.

One subject of the present invention is thus a composition comprising at least one compound of formula (I):

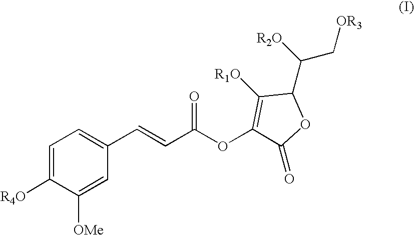

in which,

R₁ and R₄ independently represent a hydrogen atom or a group chosen from methyl, ethyl, isopropyl, ferulyl, benzyl, trimethylsilyl, triphenylsilyl, tert-butyldimethylsilyl, acetyl, propanoyl, palmitoyl, lipoyl, benzoyl, SO₃H, O=P(OH)OH and CH₃NHCO— groups,

R₂ and R₃ independently represent a hydrogen atom or a group chosen from methyl, ethyl, isopropyl, ferulyl, benzyl, trimethylsilyl, triphenylsilyl, tert-butyldimethylsilyl, acetyl, propanoyl, palmitoyl, lipoyl, benzoyl, SO₃H, O=P(OH)OH and CH₃NHCO— groups, or alternatively form, together with the oxygens to which they are attached, a 5- or 6-membered cyclic group, at least one of these two groups R₂ or R₃ being other than a hydrogen atom, and its use as an agent for bleaching the skin and/or head hair and/or other hairs.

One advantageous aspect of the invention relates to the use of the compounds of formula (I) for which R₁ represents a hydrogen atom.

In another advantageous aspect of the invention, the compounds of formula (I) used are those for which the group R₄ represents a hydrogen atom or an acetyl group.

In preferred compounds of formula (I), the groups R₂ and R₃ form, together with the oxygen atoms to which they are attached, an isopropylidene group.

One compound of formula (I) that is particularly suitable for use in the present invention is the ester 5-(2,2-dimethyl[1,3]dioxolan-4-yl)-4-hydroxy-2-oxo-2,5-dihydro-3-furanyl 3-[(4-acetoxy-3-methoxy)phenyl]acrylate of formula (A):

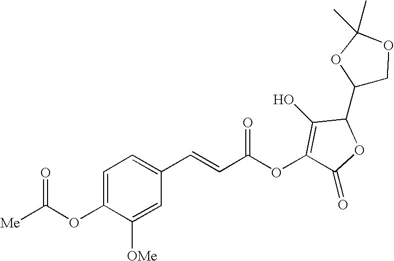

Another compound of formula (I) that is particularly suitable for use in the present invention is the ester 5-(2,2-dimethyl[1,3]dioxolan-4-yl)-4-hydroxy-2-oxo-2,5-dihydro-3-furanyl 3-[(4-hydroxy-3-methoxy)phenyl]acrylate of formula (B):

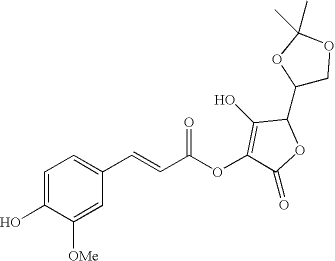

The compounds of formula (I) may be obtained by a person skilled in the art via synthesis according to usual methods, especially using the processes described in patent application EP-0 664 290. Compound A is described in the patent application in step 4 of Example 1.

The present invention also relates to the use of at least one compound of formula (I) as defined above, in a composition, as an anti-tanning agent for the skin.

The invention also relates to the use of at least one compound of formula (I) as defined above for the manufacture of a dermatological composition for depigmenting the skin and/or head hair and/or other hairs.

A subject of the invention is also a cosmetic process for bleaching human skin and/or head hair and/or other hairs, comprising the application to the skin and/or head hair and/or other hairs of a cosmetic composition containing a compound of formula (I) as defined above.

The amount of compounds that may be used in the context of the invention obviously depends on the desired effect, and is not limited.

By way of example, this amount may range, for example, from 0.001% to 10% by weight, preferably from 0.01% to 5% by weight and especially from 0.1% to 2% by weight relative to the total weight of the composition.

The composition according to the invention is especially intended for topical application. It thus preferably comprises a physiologically acceptable medium, i.e. a medium that is compatible with the skin, including the scalp, mucous membranes, head hair, other hairs and/or the eyes and may especially constitute a cosmetic or dermatological composition.

The composition may further comprise any constituent useful for the intended application.

Mention may be made especially of water, solvents, oils of mineral, animal and/or plant origin, waxes, pigments, fillers, surfactants, cosmetic or dermatological active agents, UV-screening agents, polymers, gelling agents and preserving agents.

When the composition of the invention is an emulsion, the proportion of the fatty phase is not limited and can range from 5 to 80% by weight, and preferably form 5 to 50% by weight, relative to the total weight of the composition. The oils, the emulsifiers and the co-emulsifiers used in the composition in emulsion form are chosen from those used conventionally in the field considered. The emulsifier and the co-emulsifier are present in the composition in a proportion ranging from 0.3 to 30% by weight, and preferably from 0.5 to 20% by weight, relative to the total weight of the composition.

Needless to say, a person skilled in the art will take care to select this or these optional additional compound(s), and/or the amount thereof, such that the advantageous properties of the compounds according to the invention are not, or are not substantially, adversely affected by the envisaged addition.

The composition of the invention may be in any form, including any pharmaceutical form normally used in cosmetics and dermatology, in particular in the form of an optionally gelled aqueous or aqueous-alcoholic solution, an optionally two-phase lotion-type dispersion, an oil-in-water or water-in-oil or multiple emulsion, an aqueous gel, a dispersion of oil in an aqueous phase with the aid of spherules, these spherules possibly being polymeric nanoparticles such as nanospheres and nanocapsules or better still lipid vesicles of ionic and/or non-ionic type.

The invention composition may be relatively fluid and have the appearance of a white or colored cream, an ointment, a milk, a lotion, a serum, a paste or a foam. It may optionally be applied to the skin or to head hair in aerosol form. It may also be in solid form and, for example, in the form of a stick. It can be used as a care product and/or as a make-up product. It can also be in the form of a shampoo or a conditioner.

The invention composition may constitute a cleansing, protective, treatment or care cream for the face, the hands, the feet, the major anatomical folds or the body (for example day creams, night creams, makeup-removing creams, foundation creams or antisun creams); a fluid foundation, a makeup-removing milk, a protective or care body milk or an antisun milk; a skincare lotion, gel or mousse, or as a cleansing lotion.

In one advantageous aspect of the invention, the compositions used may also comprise at least one desquamating agent, and/or at least one calmative and/or at least one organic photoprotective agent and/or at least one mineral photoprotective agent.

The term “desquamating agent” means any compound capable of acting:

either directly on the desquamation by promoting exfoliation, such as β-hydroxy acids, in particular salicylic acid and its compounds (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol;

or on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; amino-sulphonic compounds and in particular (N-2-hydroxy-ethylpiperazine-N-2-ethane)sulphonic acid (HEPES); compounds of 2-oxothiazolidine-4-carboxylic acid (procysteine); compounds of a-amino acids of the type such as glycine (as described in EP-0 852 949, and sodium methylglycinediacetate sold by BASF under the trade name Trilon M); honey; sugar compounds such as o-octanoyl-6-D-maltose and N-acetylglucosamine.

As calmatives that may be used in the composition according to the invention, mention may be made of: pentacyclic triterpenes and extracts of plants (e.g.: Glycyrrhiza glabra) containing them, for instance β-glycyrrhetinic acid and salts and/or compounds thereof (glycyrrhetinic acid monoglucoronide, stearyl glycyrrhetinate or 3-stearoyloxyglycyrrhetic acid), ursolic acid and its salts, oleanolic acid and its salts, betulinic acid and its salts, an extract of Paeonia suffruticosa and/or lactiflora, salicylic acid salts and in particular zinc salicylate, the phycosaccharides from the company Codif, an extract of Laminaria saccharina, canola oil, bisabolol and camomile extracts, allantoin, Sepivital EPC (phosphoric diester of vitamins E and C) from SEPPIC, omega-3 unsaturated oils such as musk rose oil, blackcurrant oil, ecchium oil, fish oil, plankton extracts, capryloylglycine, Seppicalm VG (sodium palmitoylproline and Nymphea alba) from SEPPIC, an extract of Pygeium, an extract of Boswellia serrata, an extract of Centipeda cunnighami, an extract of Helianthus annuus, an extract of Linum usitatissimum, tocotrienols, extracts of Cola nitida, piperonal, an extract of clove, an extract of Epilobium angustifolium, Aloe vera, an extract of Bacopa moiniera, phytosterols, cortisone, hydrocortisone, indomethacin and betamethasone.

The organic photoprotective agents are chosen especially from anthranilates; cinnamic compounds; dibenzoylmethane compounds; salicylic compounds, camphor compounds; triazine compounds such as those described in patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone compounds; β,β-diphenylacrylate compounds; benzotriazole compounds; benzalmalonate compounds; benzimidazole compounds; imidazolines; bis-benzazolyl compounds as described in patents EP 669 323 and U.S. Pat. No. 2,463,264; p-aminobenzoic acid (PABA) compounds; methylenebis(hydroxyphenylbenzotriazole) compounds as described in patent applications U.S. Pat. No. 5,237,071, U.S. Pat. No. 5,166,355, GB 2 303 549, DE 197 26 184 and EP 893 119; and screening polymers and screening silicones such as those described especially in patent application WO 93/04665; dimers derived from α-alkyl-styrene, such as those described in patent application DE 198 55 649.

The mineral photoprotective agents are chosen from pigments or nanopigments (mean size of the primary particles: generally between 5 nm and 100 nm and preferably between 10 nm and 50 nm) of coated or uncoated metal oxides, for instance nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are moreover alumina and/or aluminium stearate. Such coated or uncoated metal oxide nanopigments are described in particular in patent applications EP 518 772 and EP 518 773.

The photoprotective agents are generally present in the composition according to the invention in proportions ranging from 0.1% to 20% by weight relative to the total weight of the composition, and preferably ranging from 0.2% to 15% by weight relative to the total weight of the composition.

The examples that follow illustrate the invention without limiting its scope. Depending on the case, the compounds are cited as the chemical names or as the CTFA names (International Cosmetic Ingredient Dictionary and Handbook).

### EXAMPLE 1

### Demonstration of the Activity on Melanogenesis

A biological test demonstrated the depigmenting activity of the ascorbic acid ester compounds of formula (I).

The melanogenesis-modulating effect of the compounds of formula (I) was measured according to the method described in patent application EP-0 993 826 filed by the Applicant, and also in the article by R. Schmidt, P. Krien and M. Regnier, Anal. Biochem., 235(2), 113-18, (1996).

The following were determined for each test compound:

the cytotoxicity, by estimating the incorporation of leucine,

the inhibitory activity on melanin synthesis, by estimating the ratio of the incorporation of thiouracil to the incorporation of leucine, relative to 100% of the control (the control corresponds to the test performed without test compound),

the IC₅₀ value, which corresponds to the micromolar (μM) concentration for which a 50% inhibition of melanogenesis is observed.

The results are collated in the following table:

| | | | Test | Inhibitory |
|---|---|---|---|---|
| | IC₅₀ | Cyto- | concentration | activity on |
| | (μM) | toxicity | (μM) | melanogenesis |
| Compound T1 | No effect | No | 312 | No effect |
| Compound T2 | No effect | No | 327 | No effect |
| Compound A | 200 | No | 230 | −50% |
| Compound B | 500 | No | 262 | −60% |

Compound T1: methyl 2,3-bis-acetate of ascorbic acid
Compound T2: ascorbyl 2-cinnamate
Compounds A and B are described above.

The compounds of formula (I) are thus shown to be very effective in inhibiting melanogenesis, in contrast with the other ascorbic acid esters tested.

### EXAMPLE 2

### Demonstration of the activity on the Dendricity of Melanocytes

The aim of this test is to show the effect of the compounds used according to the invention on the morphological modulation of melanocytes.

Method:

The melanocytes are treated immediately on inoculation with the compounds according to the invention for 7 days. The compounds are used at a dose of 100 μg per ml.

Observations:

The melanocytes are labelled using the antibody NKlbeteb (green coloration), which recognizes melanosomes at any stage of maturation. The nucleus of all the cells, melanocytes and keratinocytes, is stained using propidium iodide (red coloration).

Results:

In the absence of the compounds according to the invention, the melanocytes in co-culture are highly dendritic. The pigment is visibly transferred to the neighbouring keratinocytes.

In the presence of the compounds according to the invention, most of the melanocytes show reduced dendricity and some melanocytes become bipolar.

For a higher concentration, most of the melanocytes are bipolar and the amount of pigment transferred to the keratinocytes is reduced.

The observations are summarized in the following table:

| | Test | | |
|---|---|---|---|
| Test | concentration | | Transfer of |
| compound | (μM) | Dendricity | pigmentation |
| Control | — | Multipolar | Large |
| Compound T1 | 312 | Multipolar | Large |
| Compound T2 | 327 | Multipolar | Large |
| Compound A | 230 | Bipolar | Little |
| | | | pigmentation |
| | | | transferred |
| Compound B | 262 | Bipolar | Little |
| | | | pigmentation |
| | | | transferred |

For the control, the test was performed without compound according to the invention. Compound T1: methyl 2,3-bis-acetate of ascorbic acid Compound T2: ascorbyl 2-cinnamate

Compounds A and B are described above. For these compounds, it emerges from the observations that the melanocytes become bipolar and that the amount of pigment present in the keratinocytes is reduced after treatment, in comparison with the control and the two reference compounds T1 and T2.

### EXAMPLE 3

A facial care bleaching cream of oil-in-water emulsion type is prepared, comprising (% by weight):

| | |
|---|---|
| 5-(2,2-dimethyl[1,3]dioxolan-4-yl)-4- | 0.005% |
| hydroxy-2-oxo-2,5-dihydro-3-furanyl | |
| 3-[(4-acetoxy-3-methoxy)phenyl]acrylate ester | |
| glyceryl stearate | 2% |
| Polysorbate 60 (Tween 60 from ICI) | 1% |
| stearic acid | 1.4% |
| triethanolamine | 0.7% |
| carbomer | 0.4% |
| liquid fraction of shea butter | 12% |
| perhydrosqualene | 12% |
| antioxidant | 0.05% |
| fragrance, preserving agent | qs |
| water | qs 100% |

### EXAMPLE 4

A skin depigmenting gel is prepared, comprising (% by weight):

| | |
|---|---|
| 5-(2,2-dimethyl[1,3]dioxolan-4-yl)-4- | 2% |
| hydroxy-2-oxo-2,5-dihydro-3-furanyl | |
| 3-[(4-hydroxy-3-methoxy)phenyl]acrylate ester | |
| hydroxypropylcellulose (Klucel H from Hercules) | 1% |
| antioxidant | 0.05% |
| isopropanol | 40% |
| fragrance, preserving agent | qs |
| water | qs 100% |

As noted above, the present invention relates to the use of ascorbic acid compounds, especially esters, alone or in a composition, preferably a cosmetic composition, to, e.g., bleach human skin, head hair or other hairs, protect against tanning, and/or reverse the effects of tanning. As is clear to one of ordinary skill in the art, the amount of compound/composition used is preferably at least that amount that is effective to accomplish these stated objectives.

The above written description of the invention provides a manner and process of making and using it such that any person skilled in this art is enabled to make and use the same, this enablement being provided in particular for the subject matter of the appended claims, which make up a part of the original description and including the cosmetic use of at least one compound of formula (I):

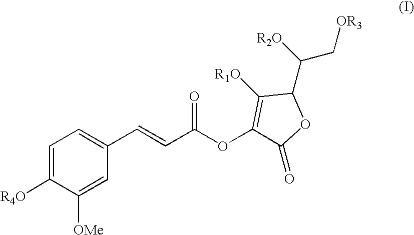

in which,

R₁ and R₄ independently represent a hydrogen atom or a group chosen from methyl, ethyl, isopropyl, ferulyl, benzyl, trimethylsilyl, triphenylsilyl, tert-butyldimethylsilyl, acetyl, propanoyl, palmitoyl, lipoyl, benzoyl, SO₃H, O=P(OH)OH and CH₃NHCO— groups,

R₂ and R₃ independently represent a hydrogen atom or a group chosen from methyl, ethyl, isopropyl, ferulyl, benzyl, trimethylsilyl, triphenylsilyl, tert-butyldimethylsilyl, acetyl, propanoyl, palmitoyl, lipoyl, benzoyl, SO₃H, O=P(OH)OH and CH₃NHCO— groups, or alternatively form, together with the oxygens to which they are attached, a 5- or 6-membered cyclic group, at least one of these two groups R₂ or R₃ being other than a hydrogen atom,

in a cosmetic composition, as an agent for bleaching the skin and/or head hair and/or other hairs. Preferred embodiments include a cosmetic process for bleaching human skin and/or head hair and/or other hairs, comprising the application to the skin and/or head hair and/or other hairs of a cosmetic composition containing at least one compound of formula (I) above.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method for bleaching skin and/or hair comprising applying thereto at least one compound of formula (I): 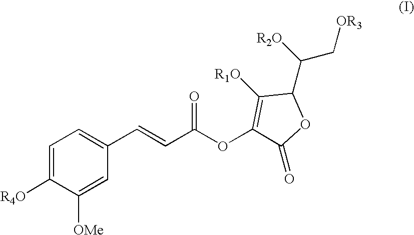
in which,
R₁ and R₄ are independently selected from the group consisting of hydrogen, methyl, ethyl, isopropyl, ferulyl, benzyl, trimethylsilyl, triphenylsilyl, tert-butyldimethylsilyl, acetyl, propanoyl, palmitoyl, lipoyl, benzoyl, SO₃H, O=P(OH)OH and CH₃NHCO— groups,and
R₂ and R₃ are independently selected from the group consisting of hydrogen methyl, ethyl, isopropyl, ferulyl, benzyl, trimethylsilyl, triphenylsilyl, tert-butyldimethylsilyl, acetyl, propanoyl, palmitoyl, lipoyl, benzoyl, SO₃H, O=P(OH)OH and CH₃NHCO— groups, or R₂ and R₃ alternatively form, together with the oxygens to which they are attached, a 5- or 6-membered cyclic group, at least one of R₂ and R₃ being other than a hydrogen atom.

2. The method according to claim 1, wherein R₂ and R₃ form, together with the oxygens to which they are attached, a 5- or 6-membered cyclic group.

3. The method according to claim 1, wherein R₁ represents a hydrogen atom.

4. The method according to claim 1, wherein R₄ represents a hydrogen atom or an acetyl group.

5. The method according to claim 1, wherein groups R₂ and R₃ form, together with the oxygen atoms to which they are attached, an isopropylidene group.

6. The method according to claim 1, wherein the compound of formula (I) is the ester 5-(2,2-dimethyl[1,3]dioxolan-4-yl)-4-hydroxy-2-oxo-2,5-dihydro-3-furanyl 3-[(4-acetoxy-3-methoxy)-phenyl]acrylate.

7. The method according to claim 1, wherein the compound of formula (I) is the ester 5-(2,2-dimethyl[1,3]dioxolan-4-yl)-4-hydroxy-2-oxo-2,5-dihydro-3-furanyl 3-[(4-hydroxy-3-methoxy)-phenyl]acrylate.

8. The method according to claim 1, wherein said compound of formula (I) is present in a composition, said composition further comprising a physiologically acceptable medium.

9. The method according to claim 8, wherein said compound of formula (I) is present in said composition in 0.001% to 10% by weight relative to the total weight of the composition.

10. The method according to claim 8, wherein said compound of formula (I) is present in said composition in 0.01% to 5% by weight relative to the total weight of the composition.

11. The method according to claim 8, wherein said compound of formula (I) is present in said composition in 0.1% to 2% by weight relative to the total weight of the composition.

12. The method according to claim 9, wherein the compound of formula (I) is the ester 5-(2,2-dimethyl[1,3]dioxolan-4-yl)-4-hydroxy-2-oxo-2,5-dihydro-3-furanyl 3-[(4-acetoxy-3-methoxy)-phenyl]acrylate.

13. The method according to claim 9, wherein the compound of formula (I) is the ester 5-(2,2-dimethyl[1,3]dioxolan-4-yl)-4-hydroxy-2-oxo-2,5-dihydro-3-furanyl 3-[(4-hydroxy-3-methoxy)-phenyl]acrylate.

14. The method according to claim 10, wherein the compound of formula (I) is the ester 5-(2,2-dimethyl[1,3]dioxolan-4-yl)-4-hydroxy-2-oxo-2,5-dihydro-3-furanyl 3-[(4-acetoxy-3-methoxy)-phenyl]acrylate.

15. The method according to claim 10, wherein the compound of formula (I) is the ester 5-(2,2-dimethyl[1,3]dioxolan-4-yl)-4-hydroxy-2-oxo-2,5-dihydro-3-furanyl 3-[(4-hydroxy-3-methoxy)-phenyl]acrylate.

16. The method according to claim 11, wherein the compound of formula (I) is the ester 5-(2,2-dimethyl[1,3]dioxolan-4-yl)-4-hydroxy-2-oxo-2,5-dihydro-3-furanyl 3-[(4-acetoxy-3-methoxy)-phenyl]acrylate.

17. The method according to claim 11, wherein the compound of formula (I) is the ester 5-(2,2-dimethyl[1,3]dioxolan-4-yl)-4-hydroxy-2-oxo-2,5-dihydro-3-furanyl 3-[(4-hydroxy-3-methoxy)-phenyl]acrylate.

18. A method for protecting the skin from tanning, or reversing the effects of tanning, comprising applying to skin at least one compound of formula (I): 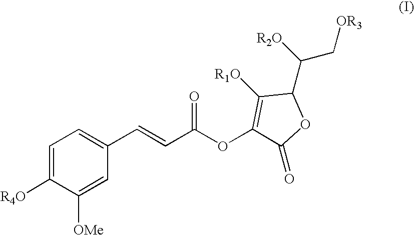
in which,
R₁ and R₄ are independently selected from the group consisting of hydrogen, methyl, ethyl, isopropyl, ferulyl, benzyl, trimethylsilyl, triphenylsilyl, tert-butyldimethylsilyl, acetyl, propanoyl, palmitoyl, lipoyl, benzoyl, SO₃H, O═P(OH)OH and CH₃NHCO— groups, and
R₂ and R₃ are independently selected from the group consisting of hydrogen methyl, ethyl, isopropyl, ferulyl, benzyl, trimethylsilyl, triphenylsilyl, tert-butyldimethylsilyl, acetyl, propanoyl, palmitoyl, lipoyl, benzoyl, SO₃H, O═P(OH)OH and CH₃NHCO— groups, or R₂ and R₃ alternatively form, together with the oxygens to which they are attached, a 5- or 6-membered cyclic group, at least one of R₂ and R₃ being other than a hydrogen atom.

19. The method according to claim 18, wherein the compound of formula (I) is the ester 5-(2,2-dimethyl[1,3]dioxolan-4-yl)-4-hydroxy-2-oxo-2,5-dihydro-3-furanyl 3-[(4-acetoxy-3-methoxy)-phenyl]acrylate.

20. The method according to claim 18, wherein the compound of formula (I) is the ester 5-(2,2-dimethyl[1,3]dioxolan-4-yl)-4-hydroxy-2-oxo-2,5-dihydro-3-furanyl 3-[(4-hydroxy-3-methoxy)-phenyl]acrylate.
